(19) United States
(12) Patent Application Publication
(10) Pub. No.: US 2005/0137560 A1
(43) Pub. Date: Jun. 23, 2005

(54) Interlabial pad

(75) Inventors: Satoshi Mizutani, Kagawa (JP); Wataru Yoshimasa, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-Shi (JP)

(21) Appl. No.: 11/007,593

(22) Filed: Dec. 8, 2004

(30) Foreign Application Priority Data
Dec. 9, 2003 (JP) 2003-411089

(51) Int. Cl. A61F 13/15; A61F 13/20

(52) U.S. Cl. 604/385.17

(57) ABSTRACT

It is an object of the present invention to provide an interlabial pad, with which regions that are exposed from the labia do not contact the groin readily and can cushion impacts even when contact occurs so that the interlabial pad will not fall off readily. An interlabial pad 20 to be fitted between the labia of a wearer is equipped with an contact avoiding means 26A and/or an impact cushioning means 26B at parts that are exposed from the labia during fitting. In the case of an interlabial pad having a shape that is contained completely between the labia, an impact cushioning means 26B is equipped.

### CROSS-REFERENCE TO RELATED APPLICATIONS

This application is based upon and claims the benefit of priority from Japanese Patent application No. 2003-411089 filed on Dec. 9, 2003, the entire contents of which is incorporated herein by reference.

1. FIELD OF THE INVENTION

The present invention concerns an interlabial pad to be fitted between the labia of a woman, and to be more detailed, concerns an interlabial pad with a structure that will not fall off even under movements of a wearer.

2. RELATED ART

Since priorly, interlabial pads which are used by being applied between labia of women have been known as menstrual products for women (see Patent Document 1). Since such an interlabial pad is used by a part thereof being sandwiched between the labia, it is put in highly close contact with a body leading to less leakage of menstrual blood and the like, in comparison to a menstrual napkin. An interlabial pad also has a merit of being low in psychological reluctance during fitting in comparison to a tampon, which is inserted inside the vagina. Meanwhile, since an interlabial pad is fitted onto a body by the sandwiching force of the labia, there is a need to prevent the interlabial pad from being fallen off the labia of the wearer. Thus various interlabial pad shapes that enable closer contact with a body to be achieved during fitting have been examined (see Patent Document 2).

The interlabial pad disclosed in Patent Document 1 has a substantially elliptical shape with protrusions disposed on a face (back side face) of a cover member opposite to a face that contacts the labia at substantially the centers in the longitudinal direction of the cover member. A wearer first pinches the protrusions and can then fit the interlabial pad in close contact with the interior of the labia upon folding the interlabial pad in two along the central part in the longitudinal direction as the axis with a face (surface) of the interlabial pad that comes in contact with the labia being the outer side.

The interlabial pad described in Patent Document 2 is substantially vertically long shape and at central parts in the longitudinal direction and lateral direction of the interlabial pad are provided pinched parts, which are pinched towards the centers in the respective directions, and by the provision of these pinched parts, the interlabial pad can be folded readily in two and be fitted readily onto the labia of a wearer.

[Patent Document 1] Japanese Translation of International Application No. H-11-500341

[Patent Document 2] United States Patent Application Publication No. 2002-0193770 Specification

However, with the interlabial pad disclosed in Patent Document 1, since only a part of the interlabial pad is held between the labia, the regions that are exposed from the labia of a wearer is readily influenced by impacts, such as pressures resulting from movements of the wearer, and there is a high possibility for the interlabial pad to fall off from the labia due to such impacts etc., being transmitted to the part that is held between the labia. Also, with the interlabial pad disclosed in Patent Document 2, though pinched parts are provided at central parts of the interlabial pad, the positions of these pinched parts are not matched to the shape of a wearer's groin. Since the interlabial pad thus contacts the part at which the width between opposing groin parts is the narrowest, the interlabial pad is readily influenced by impacts, etc., as is the interlabial pad disclosed in Patent Document 1, and is high in the possibility of falling off.

The present invention has been made in view of the above issues and an object thereof is to provide an interlabial pad with which, even when the interlabial pad contacts groin parts in accompaniment with movements of a wearer, the impact of contact can be cushioned to prevent the falling off the interlabial pad.

### SUMMARY OF THE INVENTION

In order to achieve the above object, the present invention provides an interlabial pad having a structure, with which, even when the interlabial pad contacts a body of a wearer due to movements of the wearer during fitting, the impact that caused by the contact will not be transmitted directly. To be specific, the present invention's interlabial pad is characterized in that a waist part formed by curving a part of peripheral parts or a readily compressible part is provided around the peripheral parts.

The present invention specifically provides the following:

(1) An interlabial pad being applied by inserting a part or whole thereof between labia, the interlabial pad having, at least at peripheral parts that form the contour of the interlabial pad, a contact avoiding means avoiding contacts with a wearer's body that accompanies movements of the wearer in a fitted state; and/or an impact cushioning means cushioning the transmission of impacts resulting from contacts with the body.

With the present invention described (1), a contact avoiding means to avoid contact by curving parts that contact a body, and/or an impact cushioning means which makes the parts that contact the body become compressed readily upon contact with the body, are provided at peripheral parts and vicinity thereof of an interlabial pad. When the contact avoiding means is provided, regions that are not held between the labia of a wearer can be prevented from contacting the wearer's body when the wearer moves, and impacts etc. such as pressure which are occurred when the interlabial pad contacts the body can be avoided. Meanwhile, when the impact cushioning means is provided, even if the interlabial pad contacts the wearer's body due to movements of the wearer, the resulting impact can be alleviated by the peripheral parts and vicinity thereof being compressed. Thus with the present invention, the interlabial pad can be prevented from falling off the labia of a wearer against pressure and other impacts resulting from movements of the wearer.

“Peripheral parts” refers to the outer edges and vicinity thereof of the interlabial pad of the present invention. In the case of an interlabial pad with a structure which has an absorbent body sandwiched and enclosed by two sheets, the peripheral parts are the parts positioned at the outer sides of the absorbent body at which the sheets are joined to each other without sandwiching the absorbent body. The phrase, “at least at peripheral parts that form the contour of the interlabial pad,” indicates that not only the peripheral parts but regions near the peripheral parts are also included. The “regions that are not held between the labia of a wearer” refers to the regions that are not contained within the labia but protrude outside the labia in the fitted state. These regions may be positioned in the front or rear or to the left or right side of the labia.

(2) The interlabial pad according to (1), wherein the interlabial pad has a substantially vertically long shape with a longitudinal direction and a lateral direction, the contact avoiding means comprises waist parts formed by denting parts of both sides of the peripheral parts positioned at both sides of a longitudinal central axis extending along the longitudinal direction towards the longitudinal central axis; and, in the fitted state, the waist parts are positioned at the narrowest position between legs at the groin part of the wearer.

With the present invention described (2), by “waist parts” provided at both sides of the peripheral parts that are positioned at both sides across a longitudinal central axis the interlabial pad can readily follow the shape of a wearer's labia and can be put in highly close contact with the labia. This arrangement prevents impacts caused by movements of the wearer from being transmitted to central regions of the interlabial pad and is thus effective for preventing the interlabial pad from being fallen off the wearer's labia.

Here, “both sides of the peripheral parts positioned at both sides of a longitudinal central axis” refer to regions that are positioned at the left and right sides when the interlabial pad is folded in two along the longitudinal central axis extending along the longitudinal direction. Also, “groin part” refers to the base part of the legs, and the ostium vaginae is located at the “position where the interval between legs at the groin part of a wearer is the narrowest.”

(3) The interlabial pad according to (1) or (2), wherein the impact cushioning means is arranged by a buckling strength of the peripheral parts being no less than 0.05N and no more than 0.4N.

With the present invention described in (3), by a buckling strength of the impact cushioning means being adjusted to be in a range from 0.05N to 0.4N, even when a peripheral part contacts a groin part, etc., of a wearer in the fitted state, the interlabial pad itself can buckle in part and absorb the impact. The impact that arises from contact with the vulva or groin part, etc., is thus made less likely to be transmitted to a part of the interlabial pad held between the labia. Here, “buckling strength” refers to the readiness of the peripheral parts and vicinity thereof to become curved (compressed) in response to an impact (pressure) that is applied from a face at an end face (in the thickness direction) of a peripheral part. The method of measurement shall be described later. If this buckling strength is less than 0.5N, since the peripheral parts and vicinity thereof will be more readily compressed and curved than necessary, the peripheral parts will curl up to form a bulk in the fitted state and thus add a foreign-body sensation to the fitting feeling. Meanwhile, if the buckling strength is greater than 0.4N, since the peripheral parts will not deform when the end faces of the peripheral parts contact the skin, the wearer's skin may become damaged. In regard to this point, with the buckling strength of the present invention, the falling-off of the interlabial pad can be prevented without adverse effects on the fitting feeling.

(4) The interlabial pad according to any of (1) to (3), wherein the impact cushioning means comprises a plurality of incisions that are provided in the interlabial pad.

“Incisions” refer to cuts that are made in the peripheral parts or vicinity thereof. Though these incisions are preferably provided at a part of the peripheral parts that are positioned at side parts of the interlabial pad, they may be provided in the entirety of the peripheral parts. For example, in the case of an interlabial pad comprising a surface side sheet which contacts the body, a back side sheet which is positioned at the side opposite the surface side sheet, and an absorbent body, the peripheral parts may be provided in either of two patterns described hereinafter. That is, there may be peripheral parts that are formed by the joining only the surface side sheet and the back side sheet, or there may be peripheral parts that are formed by the joining the surface side sheet, the back side sheet, and the absorbent body. With these cases, the incisions provided in the peripheral parts may take on one of the three patterns; in the first pattern the incisions are provided in the surface side sheet, back side sheet, and the absorbent body, in the second pattern the incisions are provided in just the absorbent body, in the third pattern the incisions are provided in just the surface side sheet and the back side sheet. The direction of the incisions is not restricted and may be longitudinal, lateral, diagonal, or incisions of such directions may be mixed. The shape of the incisions is also not restricted in particular and may be linear, curved, wave-like, etc. The peripheral parts can be formed by only the surface side sheet or only the back side sheet.

(5) The interlabial pad according to (4), wherein at least a part of the incisions are provided so as to be parallel to the lateral direction of the interlabial pad.

With the present invention described in (5), the incisions that are provided so as to be parallel to the lateral direction of the interlabial pad can open up in the longitudinal direction of the interlabial pad when an external pressure, resulting from a movement of a wearer, is applied. The peripheral parts and vicinity thereof can thereby become compressed and the external pressure can be cushioned more effectively.

(6) The interlabial pad according to any of (1) to (5), wherein the outer edges of the peripheral parts are wave-like in form.

With the present invention described in (6), since the outer edges of the peripheral parts are wave-like in form and since an impact from a groin part, etc., will thus concentrate at peak parts of wave-like shapes that is narrowed towards the end and contact to the body with small area, the peak parts and vicinity thereof can be compressed readily.

(7) The interlabial pad according to any of (1) to (6), wherein the interlabial pad is equipped with a cover member comprising a surface side sheet forming a surface side face that contacts the body in the fitted state, and a back side sheet forming a back side face at the side which is opposite to the surface side face, and the impact cushioning means comprises discontinuously joined parts at which the surface side sheet and the back side sheet are joined discontinuously.

With the present invention described in (7), since the surface side sheet and the back side sheet are joined discontinuously, regions joined by weak force and regions joined by strong force are formed. Thus even when an external force is directed from an end part of a peripheral part towards the inner side of the interlabial pad, the peripheral parts and vicinity thereof can be compressed readily towards the central region of the interlabial pad from the regions joined by weak force or the boundaries between the regions joined by weak force and the regions joined by strong force.

“Discontinuously joined parts” refer to parts at which a joined part wherein the surface side sheet and the back side sheet are joined, and a non-joined part wherein the sheets are not joined, alternate. Such the joined part and the non-joined part are repeated at least two times. In this case, a joined part is positioned at a location closest to the absorbent body and a non-joined part is positioned at a location furthest from the absorbent body. Thus in the case where the joined part and the non-joined part are repeated in from a portion of a peripheral part which is close to the absorbent body towards an end portion (outer) of a peripheral part, the location close to the absorbent body will be a joined part and the location furthest from the absorbent body will be a non-joined part.

When joined parts and non-joined parts alternate along the peripheral parts, the pitch of the joined parts is preferably in the range of 1 mm to 20 mm and more preferably in the range of 8 mm to 20 mm. Also, the joined area percentage in this case is preferably 1% to 50% and more preferably 3% to 30%. In regard to this point, when the pitch of the joined parts is greater than 20 mm or the joined area percentage is less than 1%, not only may the surface side sheet and the back side sheet peel off and the absorbent body be exposed during fitting of the interlabial pad but since the interlabial pad will be more compressible and bendable than necessary, the peripheral parts will curl up. On the other hand, if the pitch of the joined parts is less than 1 mm or the joined area percentage is greater than 50%, the peripheral parts will be rigid and the end faces will not deform even upon contact with the skin and will readily transmit an external force to the absorbent body.

The force joining the peripheral parts that contact the hypersensitive clitoris or anus in the fitted state is preferably weaker than the force joining other regions. In particular, with an interlabial pad, which is used upon being folded in two along the longitudinal central axis of the interlabial pad as the folding axis so that the back side sheet faces itself, since the buckling strength is increased by the folding in two, the force joining the peripheral joined parts is preferably weakened in advance. More preferably, incisions are made in these parts.

As a specific method of joining the surface side sheet and the back side sheet, use of an adhesive agent, emboss sealing, or other known art is employed.

As an example of adhesive agent coating patterns, a form in which a hot-melt pattern is sandwiched between the surface side sheet and the back side sheet may be cited, and a coating method using a spray gun is preferable.

Though spiral coating, controlled seam coating, coater coating, curtain coater coating, spray gun coating, etc., may be cited as methods of forming adhesive agent coating patterns, among these, spray gun coating, which enables the pitch of the joined parts and non-joined parts to be made fine, is preferable.

The basis weight per unit area of the adhesive agent is in the range of 1 to 30 g/m² and more preferably in the range 3 to 1 g/m². In the case of a pattern in which the adhesive agent is coated in a line-like manner, the line diameter is preferably in the range of 30 to 300μm. If the basis weight per unit area is less than 1g/m² or the line diameter is less than 30μm and the surface side sheet is arranged as a fiber aggregate, the adhesive agent will become buried between fibers and an adequate joining force will not be provided. On the other hand, if the basis weight per unit area is greater than 30g/m² or the line diameter is greater than 300μm, the peripheral parts will be rigid.

An emboss pattern of emboss sealing (for example, the form of embossing of the surface side sheet and the back side sheet together) is not restricted in particular and may be lattice-like, dot-like, wave-like, etc.

(8) The interlabial pad according to any of (1) to (6), wherein the interlabial pad is equipped with a cover member comprising a surface side sheet, forming a surface side face that contacts the body in the fitted state, and a back side sheet forming a back side face which is opposite to the surface side face; and the impact cushioning means includes microprotrusion regions formed of a set of microprotrusions disposed on the surface side sheet and/or the back side sheet.

“Microprotrusions” refer to shapes that protrude partially from the reference planes of the respective sheets. By providing microprotrusions on both or one of the opposing faces of the respective sheets and then joining the sheets, discontinuously joined faces are formed. The faces at which the respective sheets oppose each other are thus made high in the variation rate of unevenness, and even if an external force or other impact is applied, the peripheral parts and vicinity thereof will become compressed readily and the impact will thereby be cushioned. For example, in the case of an interlabial pad comprising a surface side sheet, an absorbent body, and a back side sheet and wherein the surface side sheet and the back side sheet are joined outside the absorbent body, by making one face of the surface side sheet that contacts the absorbent body (back side) higher in the variation rate of unevenness than the other face (surface side) opposite to the back side, that is, by making the degree of freedom of the fibers that make up the back side face high, the force joining the surface side sheet and the back side sheet can be made uniform and peripheral parts with bulkiness can be formed. Thus even when pressure is applied from end faces (in the thickness direction) of the interlabial pad, since the fibers of high degree of freedom can fluctuate, the peripheral parts will become compressed readily towards the center of the labia.

The form of the microprotrusions is not restricted in particular and the microprotrusions may be embossed parts formed by embossing, or the back side of the surface side sheet may be made a fluffed face. Since a fluffed face is high in the degree of freedom among fibers, even when pressure or other impact is applied, the fibers of high degree of freedom can fluctuate and the peripheral parts and vicinity thereof can become compressed readily. Also, the term, “set,” is used since such a shape does not function solitarily to form discontinuously joined faces. It is sufficient that the microprotrusion regions be provided at locations at which the surface side sheet and the back side sheet are joined discontinuously, and for example, may be provided just at the peripheral parts or may be provided across entire sheets. The method of joining the surface side sheet and the back side sheet is not restricted in particular, and patterned adhesive agent application, embossing, ultrasonic processing, etc., may be employed.

(9) The interlabial pad according to (8), wherein the mutual interval between the microprotrusions is no less than 0.3 mm and no more than 20 mm.

When the mutual interval between the microprotrusions next to each other is less than 0.3 mm, the peripheral parts will curl up due to the peripheral parts and vicinity being more compressible and bendable than necessary. On the other hand, if the interval is greater than 20 mm, the peripheral parts will be rigid. In regard to this point, with the present invention described in (9), the mutual height of the microprotrusions is preferably within the range of 0.3 to 2 mm. If the height is less than 0.3 mm, the peripheral parts will be rigid, and if the height is greater than 2 mm, the peripheral parts will curl up due to the peripheral parts and vicinity being more compressible and bendable than necessary.

(10) The interlabial pad according to any of (1) to (9), wherein the interlabial pad is equipped with a contained part, which is held between the labia in the fitted state, and exposed side regions, which are exposed from at least one side of the labia; and the contact avoiding means and/or the impact cushioning means are provided at the exposed side regions.

With the present invention described in (10), the contact avoiding means and/or the impact cushioning means are provided at the exposed side regions, which are most readily put in contact with the end parts of the groin, vulva, etc., by movements of a wearer. Thus even if an exposed side region contacts a wearer's leg, etc., the resulting impact can be prevented from being transmitted as it is to the contained region. Especially in regard to the contact avoiding means, in a case where waist parts are formed as in the case described in (2), by the waist parts being positioned at the exposed side regions, contact with a groin part is more readily avoided and an impact due to contact can be prevented in advance from becoming transmitted to the contained region.

(11) The interlabial pad according to (10), wherein the impact cushioning means is arranged by a buckling strength of the above-mentioned exposed side regions being no less than 0.1N and no more than 5.0N.

With the present invention described in (11), the exposed side regions are made to have a predetermined buckling strength. Here, if the buckling strength is less than 0.1N, since the regions will be more compressible than necessary, menstrual blood that had been absorbed once will flow back and be released. On the other hand, if the buckling strength is greater than 5N, the impact from the groin part cannot be cushioned adequately and this impact will be transmitted to the contained region, thereby causing the interlabial pad to fall off from between the labia. In regard to this point, the present invention is arranged to maintain a favorable fitting state without giving rise to such inconveniences.

(12) The interlabial pad according to any of (1) to (11), wherein the interlabial pad is used for absorbing vaginal discharge or for incontinence.

With the present invention described in (12), the interlabial pad of (12) can be used for absorbing vaginal discharge and/or for continence. That is, since the interlabial pad is used upon being sandwiched between the labia, it can be used to absorb secreted matter, besides menstrual blood, from the ostium vaginae. Also, since not only the ostium vaginae but the urethral meatus is also positioned between the labia, urine can be absorbed as well.

As described above, the present invention provides an interlabial pad, with which even when external pressure or other impact is applied to the interlabial pad, the impact can be avoided and/or cushioned. Leakage of menstrual blood due to the falling off of the interlabial pad as a result of movements of a wearer during use can thus be prevented. Also by the provision of the contact avoiding means and/or impact cushioning means, even when the peripheral parts contact the inner walls of the labia, flawing of the labial inner walls can be prevented.

### BRIEF DESCRIPTION OF THE DRAWINGS

FIG. 1 is a perspective front view showing an interlabial pad of a first embodiment of the present invention.

FIG. 2 is a perspective sectional view showing the interlabial pad of the first embodiment of the present invention.

FIG. 3 is diagrams showing a woman's labia.

FIG. 4 is a plan view showing the fitted state of the interlabial pad of the first embodiment of the present invention.

FIG. 5 is a sectional view showing the fitted state of the interlabial pad of the first embodiment of the present invention.

FIG. 6A is a plan view showing the fitted state of the interlabial pad of the first embodiment of the present invention.

FIG. 6B is a plan view showing the fitted state of the interlabial pad of the first embodiment of the present invention.

FIG. 6C is a plan view showing the fitted state of the interlabial pad of the first embodiment of the present invention.

FIG. 7 is a plan view showing the interlabial pad of the first embodiment of the present invention.

FIG. 8 is a sectional view showing an interlabial pad of a second embodiment of the present invention.

FIG. 9 is a plan view showing an interlabial pad of a third embodiment of the present invention.

FIG. 10 is a perspective front view showing the interlabial pad of the third embodiment of the present invention.

FIG. 11 is a perspective sectional view showing the interlabial pad of the third embodiment of the present invention.

FIG. 12 is a plan view showing an interlabial pad of a fourth embodiment of the present invention.

FIG. 13 is a sectional view showing an interlabial pad of a fifth embodiment of the present invention.

FIG. 14 is a perspective front view showing an interlabial pad of a sixth embodiment of the present invention.

FIG. 15 is a front view showing the fitted state of an interlabial pad of a seventh embodiment of the present invention.

FIG. 16 is a front perspective view of a sample in the process of measuring the buckling strength.

FIG. 17 is a stress versus strain curve of the sample as determined by buckling strength measurement.

### DESCRIPTION OF THE PREFERRED EMBODIMENT

The present invention shall now be described more specifically. In the following description of the present invention's embodiments, the same components shall be provided with the same symbols and description thereof shall be omitted or simplified.

The interlabial pads of the embodiments are prepared in reference to average dimensions of women in their twenties to fifties. Specifically, the interlabial pads were prepared based on a BMI value of 21 (=weight kg/(height m)2) and distances between opposing groin parts in an erect posture with the inner interval between knees being 35 cm are 60 mm at the anterior labial commissure, 38 mm at the clitoris, 34 mm at the ostium vaginae, and 50 mm at the anus. These values were determined by drawing a horizontal line at each part (anterior labial commissure, clitoris, etc.,) in the longitudinal direction of the body and measuring the total distance between the deepest points of the groin that intersect with the horizontal line.

Interlabial pads equipped with an impact avoiding means and/or impact cushioning means at exposed side regions are explained as a first and second embodiment of the invention.

[First Embodiment]

<Basic Structure>

First, the basic overall structure of the present invention's interlabial pad shall be described. An interlabial pad (referred to hereinafter as “pad”) 20 of a first embodiment of the present invention is a combination type provided with both a contact avoiding means and an impact cushioning means.

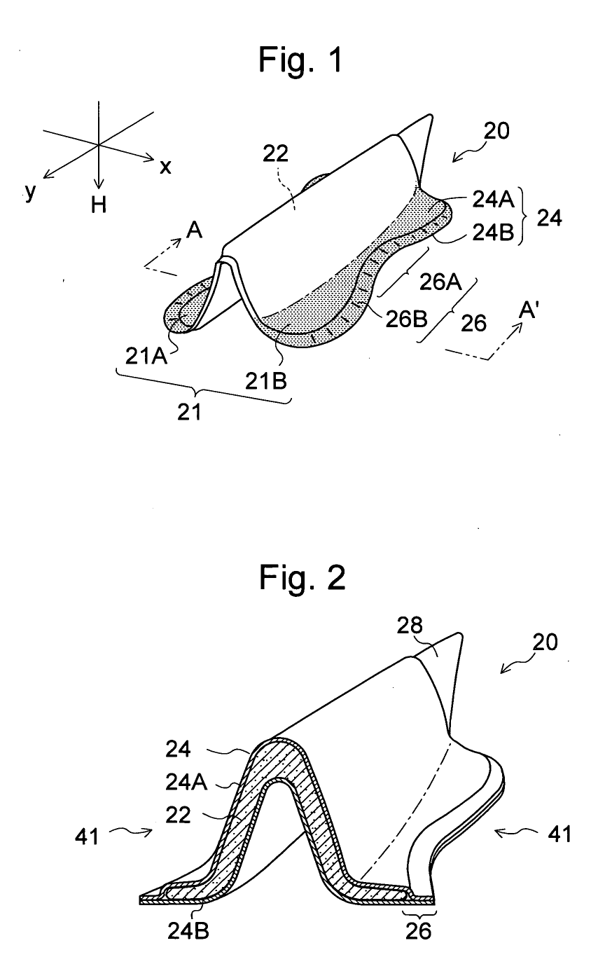

As shown in FIG. 1, this embodiment's combination type interlabial pad has an elongate shape, that is, a substantially gourd-like shape with the major diameter along the Y-axis and the minor diameter along the X-axis as viewed from directly above. However, this shape may be elliptical, gourd-like, teardrop-like, etc., and is not restricted in particular as long as it is a shape that fits the labia of women and changes in shape during fitting. Furthermore, though the pad 20 of this embodiment is of a type that is used upon being folded in two along a longitudinal central axis as the folding axis, the present invention is not restricted to this type.

The pad 20 has an absorbent body 22 provided along the longitudinal central axis, and a cover member 24 provided above and below this absorbent body 22. The cover member 24 comprises a surface side sheet 24A and a back side sheet 24B. The raw material of the surface side sheet 24A is not restricted in particular as long as it is liquid-permeable and allows the permeation of body fluids of a wearer, and the raw material of the back side sheet 24B is not restricted in particular as long as it is liquid-impermeable and practically does not allow the permeation of body fluids of a wearer. Also, the surface side sheet 24A and the back side sheet 24B enclose the absorbent body 22. Though end parts of the absorbent body 22 may be sandwiched by and joined to these sheets 24A, 24B, these end parts are preferably not sandwiched by and joined to the sheets 24A, 24B. Furthermore at the surroundings of the absorbent body 22 are provided peripheral parts 26, comprising the surface side sheet 24A, the back side sheet 24B, and the outer edges of the absorbent body 22. The width of the peripheral parts 26 is preferably 3 mm to 10 mm and more preferably 4 mm to 7 mm. A contact avoiding means 26A and an impact cushioning means 26B are provided at the peripheral parts 26. The size of region that is exposed from the labia is preferably arranged to be smaller than the distances between opposing groin parts determined by the above-described measurements, in particular, the length of outer edge is preferably arranged to be approximately 2 mm to 10 mm smaller than the distances between opposing groin parts determined by the above-described measurements.

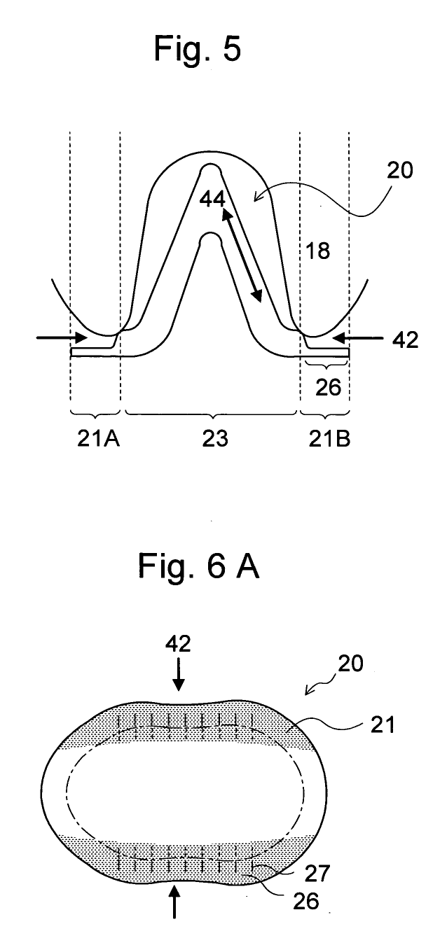

Parts of the pad 20 also have exposed side regions 21, which are exposed to the left and right from the sides of the labia. In the figure, the exposed side regions 21 are indicated as left exposed side region 21A and a right exposed side region 21B. Here, since the depth of the labia is 14 mm on the average, the region within approximately 14 mm from the longitudinal central axis which is extending along an axis H that extends perpendicularly to the longitudinal central axis of the absorbent body 22, is not exposed from the labia. Thus the parts that are positioned more than 14 mm from the longitudinal central axis contacting the vestibular floor in the fitted state are the exposed side regions, and in the present embodiment, these are the regions that extend to the left and right from inside the labia so as to cover the ends of the labia. The other regions than the exposed side regions 21 are a contained region 23 which are contained inside of the labia (see FIG. 5). The region of the absorbent body 22 which is positioned around the longitudinal central axis contacts a wearer's vestibular floor in the fitted state.

The length of the labia is 55 mm on the average and is 50 mm long in front of the ostium vaginae and 5 mm long to the rear of the ostium vaginae. Thus the “regions that are not held by the labia” in the longitudinal direction are the region that extends forward beyond 50 mm to the front of the ostium vaginae and the region that extends rearward beyond 5 mm to the rear of the ostium vaginae.

The cover member and the absorbent body, which are the most basic components of the present invention's interlabial pad, shall now be described.

FIG. 2 shows a perspective sectional view along line A-A′ of the pad 20 of FIG. 1. The absorbent body 22 has a gourd-like shape with the total length in the longitudinal direction being 90 mm, and the total length in the lateral direction being 60 mm at the maximum and 50 mm at the minimum. The bulk height is approximately 5 mm. As with the absorbent body 22, the surface side sheet 24A and the back side sheet 24B have gourd-like shapes, and both the surface side sheet 24A and the back side sheet 24B have the same size, or the surface side sheet 24A is larger by an amount corresponding to the bulk of the absorbent body 22. As specific total dimensions, the length in the longitudinal direction is 110 mm, and the length in the lateral direction is 65 mm at the maximum and 59 mm at the minimum. Also, at the end parts at one side (the parts toward the front as viewed from a wearer) in the longitudinal direction of the surface side sheet 24A and the back side sheet 24B, a knob 28, which extends in an elongated manner to enable being picked up readily, may be provided. The knob 28 are approximately 20 mm in length. The peripheral parts 26 are provided at the periphery of the absorbent body 22 and the width thereof is approximately 6 mm. Also, the apparent dimension in the lateral direction is 38 mm at the maximum and 32 mm at the minimum. The interlabial pad 20 is preferably fitted on so that the region of the minimum dimension is positioned so as to contact the ostium vaginae. Here, an “apparent dimension” refers to a minimum distance dimension of a straight segment that joins parts at both ends.

The absorbent body 22 is formed using pulp, chemical pulp, rayon, acetate, natural cotton, polymer absorbent body, or synthetic fibers, etc., in solitary form or as a mixture and is preferably bulky, lasting in form, and low in chemical irritability.

For the surface side sheet 24A, a raw material that is water-permeable, liquid-hidrophilic, and does not irritate the skin is used. A single type of non-woven fabric obtained by point bonding, air through method, or other manufacturing method or composite material of such fabrics can be cited as examples of such a raw material. Among such materials, an arrangement, having at least cellulose-based, liquid-hidrophilic fibers as the principle component, is preferable in consideration of the compatibility with the inner labial walls so that a wearer will not feel a discomfort due to deviation of the interlabial pad with respect to the inner labial walls. Specifically, a spun-lace non-woven fabric, prepared by using fibers obtained by mixing 5 to 30% natural cotton with 70 to 95% rayon or acetate, adjusting the basis weight per unit area of the fibers to within the range of 20 to 50 g/m², and entangling the fibers with each other by hydroentanglement, and then drying and adjusting the thickness to within the range of 0.3 to 1.0 mm, is preferable. In regard to the quality of the fibers to be used, natural cotton of a fiber length in the range of 15 to 60 mm and rayon or acetate of a fiber length in the range of 25 to 51 mm and a fineness in the range of 1.1 to 6.6dtex are selected. A film with permeabilize pores or an arrangement, wherein a film is laminated onto a fiber layer and permeabilize pores are provided, may also be used.

As the back side sheet 24B, a liquid-impermeable sheet, which can prevent the menstrual blood that is held in the absorbent body from leaking out of the interlabial pad, can be used. Also, by using a moisture-permeable material, accumulation of moisture during fitting can be alleviated and discomfort during fitting can thus be lightened. Examples of a material for such a liquid-impermeable sheet include polyethylene, polypropylene, polyethylene terephthalate, polyvinyl alcohol, polylactic acid, polybutyl succinate, non-woven fabric, paper, and laminates of such materials with a thickness of 15 to 60 μm. An air-permeable film, obtained by drawing a composite sheet of a resin and an inorganic filler, may also be used. A film, having low-density polyethylene (LDPE) resin as the principal component, and pores of a pore diameter of 0.1 to 0.6 mm at a porosity of 10 to 30%, and being adjusted in basis weight per unit are to within the range of 15 to 35 g/m², can be cited as a specific example. Spun-bonded non-woven fabrics, point-bonded non-woven fabrics, through-air non-woven fabrics, etc., can be cited as examples of non-woven fabrics and these may be subject to a water-repellent treatment. Among these, a SMS non-woven fabric with a three-layer of spun-bonded/melt-blown/spun-bonded (SMS) arrangement, especially being formed of ultrafine fibers and including a melt-blown layer of extremely small interfiber distance, is preferable. In this case, the layers are preferably arranged to have basis weights per unit area in the ranges of 5 to 15 g/m², 1 to 10 g/m², and 5 to 10 g/m², respectively.

<Contact Avoiding Means and Impact Cushioning Means>

The contact avoiding means and the impact cushioning means of the present embodiment shall now be described. Though with the present embodiment, both a contact avoiding means and an impact cushioning means are provided in combination, just one of either may be provided instead.

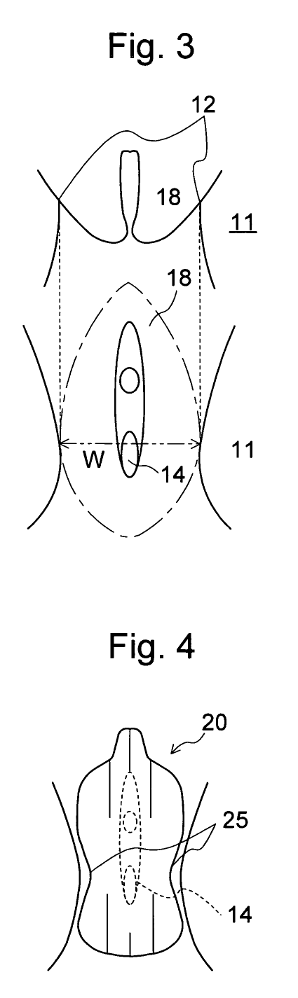

The “contact avoiding means” refers to a shape by which a part of the peripheral parts of the pad are prevented from contacting the groin part of a wearer. FIG. 3 shows a woman's labia containing a front view of the labia and a view of the labia as viewed upwards from below. As shown here, the location, at which the width W between groin parts 12 positioned at the bases of the left and right legs 11 is the narrowest, is positioned along a line that traverses the ostium vaginae 14 that is positioned at the dorsal side of the pubic region. Thus if the interlabial pad has a shape that will not contact the groin parts 12 at this location, an external pressure 41 can be prevented from being applied to the end face (thickness direction) of peripheral parts 26 of the pad 20 (see FIG. 2). That is, with the present embodiment, the forming a contact avoiding means refers to the provision of waist parts 25 at the respective sides of the part of the pad 20 that contacts the ostium vaginae 14.

Also, “impact cushioning means” refers to a means for alleviating impacts caused by movements of a wearer. Since as shown in FIG. 5, with the pad 20, the pad 20 is held between the labia 18, a force (referred to hereinafter as “external force A”) 42, which acts in the direction of the center of the pad 20, acts on exposed side regions 21, including the peripheral parts 26 of the pad 20, as a result of movement of the wearer. Also, to the contained region 23 of the pad 20, which is contained between the labia 18, a force (referred to hereinafter as “external force B”) 44 is applied due to movement of the wearer including the twisting and untwisting resulting from such movement. That is, when an external force A 42 is transmitted to the contained region 23, it becomes an external force B 44 and this causes pad 20 to fall off from the labia 18. An impact cushioning means that cushions the external force A 42 at the exposed side regions 21 is thus needed.

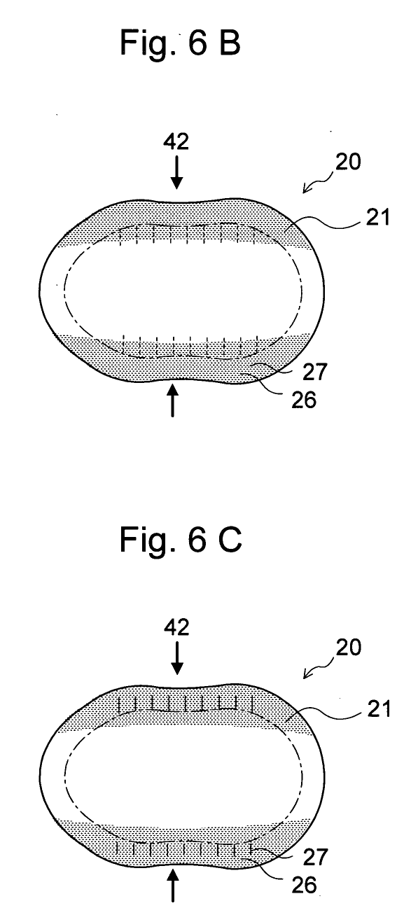

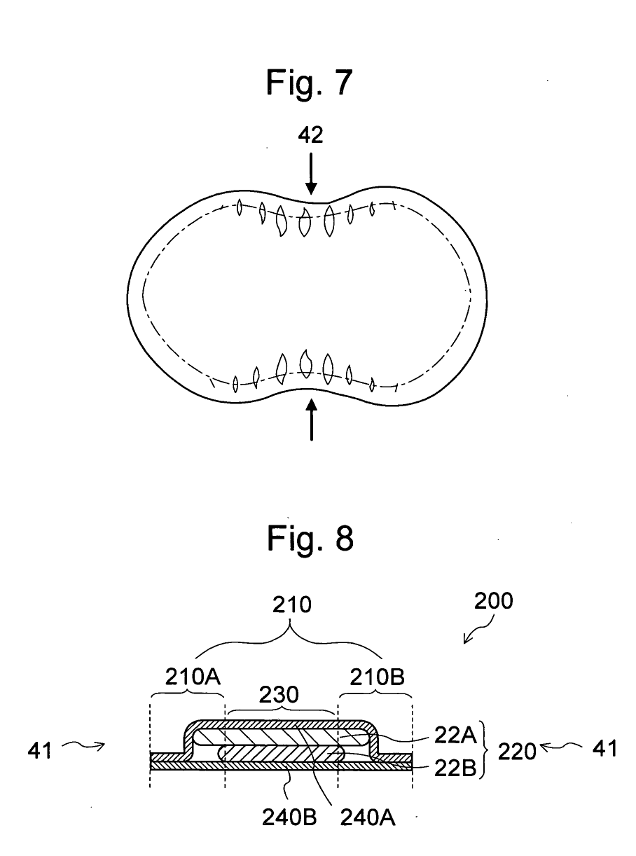

The impact cushioning means of the present embodiment refers to incisions 27, which are provided in the exposed side regions 21 and vicinity thereof as shown in FIGS. 6A to 6C. These incisions 27 are provided so as to be parallel to the external force A 42 and the mutual interval between the incisions 27 is preferably 1 mm to 20 mm and the pitch is preferably not over 20 mm. The pattern of the incisions 27 is not restricted in particular and may be formed in the longitudinal direction, lateral direction, diagonal direction, or a combination of these, and the shape of the incisions may be linear, curved, wave-like. Though the depth of incisions 27 is not restricted in particular, the incisions 27 spread more readily the deeper they are. By spreading in response to an external force A 42 as shown in FIG. 7, these incisions 27 can cushion the external force A 42.

In the case where the exposed side regions 21 contain the absorbent body and the sheets that cover the absorbent body, etc., the incisions may be formed in onlyt the absorbent body or only the sheets as shown in FIG. 6B or FIG. 6C or may be formed in both the absorbent body and the sheets.

[Second Embodiment]

With this embodiment, the buckling strength of the exposed side regions is adjusted to within a predetermined range by the arrangement of the absorbent body and selection of the raw material of the absorbent body.

With a pad 200 of this embodiment, an absorbent body 220 has a two-layered structure with an upper layer 22A and a lower layer 22B as shown in FIG. 8. By this arrangement, lower layer 22B does not exist at the exposed side regions 210 and when an external pressure 41 is applied, the exposed side regions 210 become compressed readily. In regard to the component materials, rayon with a fineness of 1.1 to 6.6dtex and fiber length of 20 to 51 mm may be layered at a basis weight 180 g/m² at upper layer 22A which comes into contact with surface side sheet 240A and pulp may be layered at 80 g/m² at the region of lower layer 22B that is to be contained between the labia. The regions where the lower layer 22B exists are contained region 230 and the regions where the lower layer 22B is absent are exposed side regions 210 as fore the pad 200. The exposed side regions 210 contain a left exposed side region 210A and a right exposed side region 210B.

In regard to the method of preparing the absorbent body 220, a sheet, formed by the air laid method, melt blown method, spun lacing method, papermaking method, etc., may be used upon embossing by passing between rolls of dot-form, lattice-form, etc. In this case, the embossed area percentage is preferably not over 20% in the regions that are exposed from the labia and preferably within the range of 0.3 to 40% in the region that is contained between the labia. The absorbent body 220 may also be subject to slitting or linear embossing in order to make it bend and be compressed readily.

Interlabial pads having impact cushioning means provided at the peripheral parts are explained as a third to seven embodiments of the invention.

[Third Embodiment]

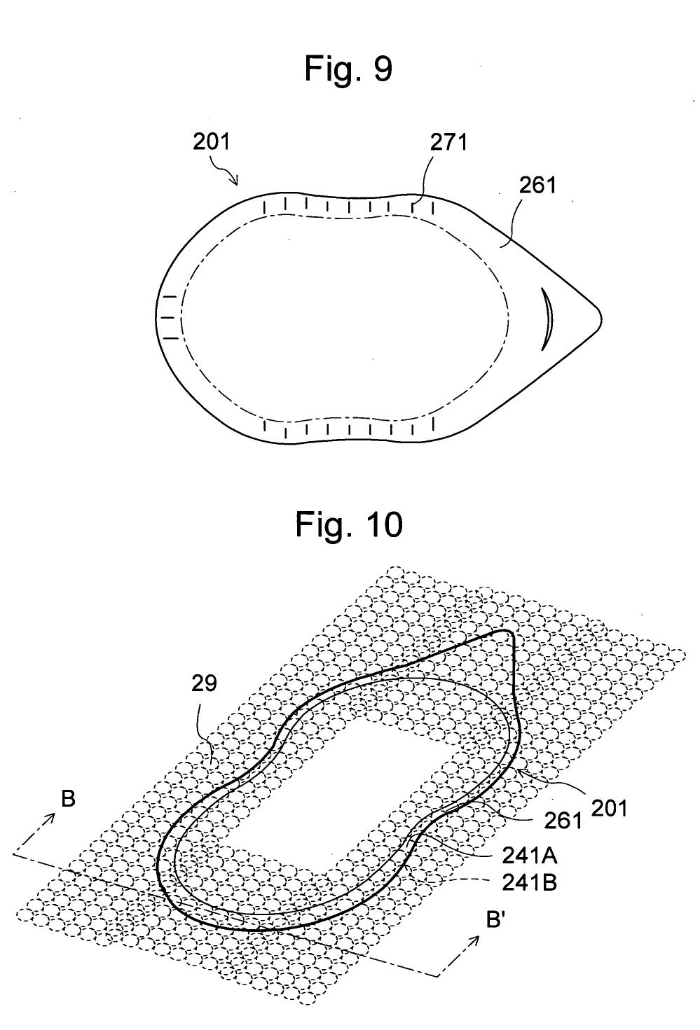

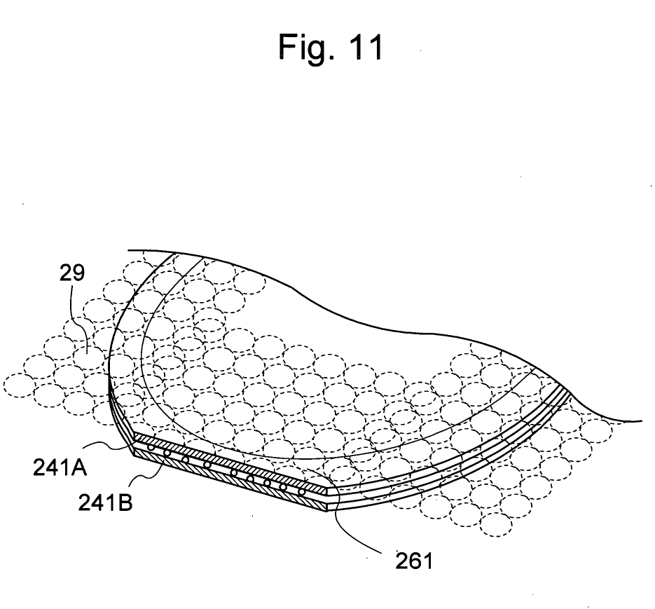

With a pad 201 of this embodiment, incisions 271 are made in the peripheral parts. Besides being made in parallel to the lateral direction, the incision pattern may also be provided in the longitudinal direction in the manner of the incisions 271 in the peripheral parts 261 of the pad 201 shown in FIG. 9. Furthermore, the incisions preferably do not reach the outer edges of the peripheral parts as a foreign-body sensation will be less likely to be caused in this case. Also, the present embodiment is an example wherein discontinuously joined parts are provided around the peripheral parts 261 of the pad 201 as an impact cushioning means by a hot melt pattern. As shown in FIG. 10, as the hot melt pattern 29, a 100% solids adhesive agent, having a thermoplastic synthetic resin or rubber as the base material, is used to form a pattern in which rings of the adhesive agent are connected in a sheet-like manner. This pattern is applied between the surface side sheet 241A and the back side sheet 241B to adhere the sheets and form locations at which the surface side sheet 241A and the back side sheet 241B are adhered and locations at which the sheets are not adhered (see FIG. 11). Preferably, the end parts of the peripheral parts 261 and the region of pad 201 that is held between the labia are not adhered by the hot melt pattern 29. By using the hot melt pattern 29, the surface side sheet 241A and the back side sheet 241B can be adhered securely and yet an appropriate adhesive force can be maintained. With the present embodiment, the hot melt pattern 29 and the incisions can be used in combination, and by combined use, the interlabial pad is made less likely to be influenced by external forces.

[Fourth Embodiment]

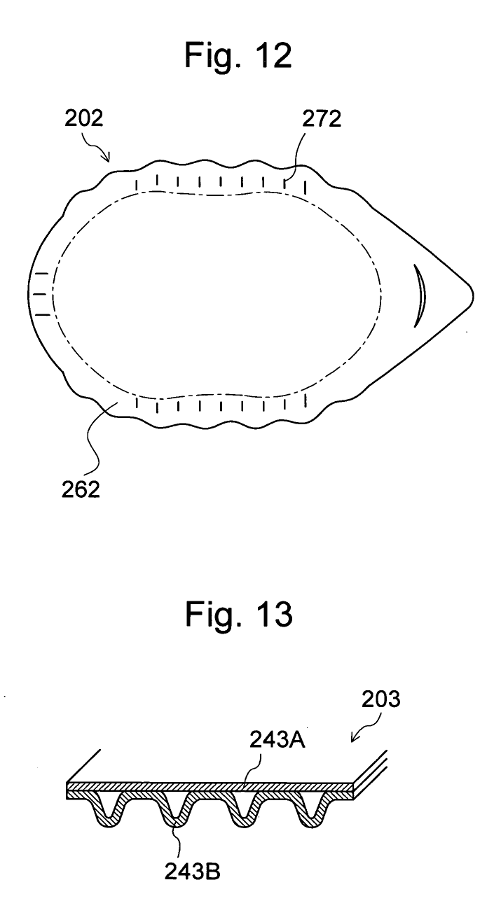

With this embodiment, outer edges, which form the contour of the interlabial pad 202, are made wave-like in form. The area that comes in contact with the body of the wearer is thus lessened and the peripheral parts 262 are made readily compressible. As a result, substantially the same effects as those of the incisions 271 of the third embodiment can be provided (see FIG. 12).

[Fifth Embodiment]

This embodiment is a microprotrusion type interlabial pad 203, which uses a back side sheet 243B having microprotrusions as the impact cushioning means. As shown in FIG. 13, the back side sheet 243B is provided with microprotrusions and the protruding parts are adhered to the surface side sheet 243A. The distance between the protruding parts is 10 mm and the joined area percentage is 20%. There is no need to provide the microprotrusions over the entire face of the back side sheet 243B and the microprotrusions may be provided just in the peripheral parts. Also the microprotrusions can be used in combination with incisions as in the second embodiment. Unlike the hot melt pattern of the second embodiment, since an adhesive agent is applied only to the protruding faces with the microprotrusions of the present embodiment, the peripheral parts can be kept in a state of low buckling strength.

[Sixth Embodiment]

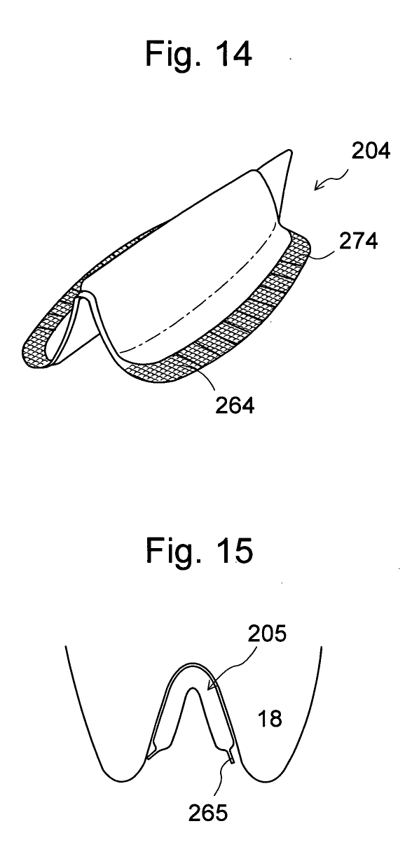

This embodiment is an interlabial pad 204 wherein the contact avoiding means is eliminated and, as the impact cushioning means, uneven shapes are formed by embossing. Embossing refers to the process of forming uneven patterns on paper, cloth, leather, etc., by the pressing of rolls, on which patterns are engraved, while heating. Though the emboss pattern of the present embodiment is not restricted in particular and may be lattice-like, flat, etc., an emboss pattern, such as a lattice-like pattern, with which the end faces are uneven and thus less likely to be sharp, will be less likely to give rise to a foreign-body sensation. FIG. 14 shows a pad 204, with which uneven shapes are formed by embossing and incisions 274 are furthermore formed at the peripheral parts 264. Since in the case of embossing, the peripheral parts 264 tend to become rigid, it is preferable to provide the incisions 274 in addition to applying embossing. Impacts can thus be cushioned more definitely while making a wearer less likely to feel foreign-body sensations during fitting.

[Seventh Embodiment]

This embodiment is a contained type interlabial pad 205 with which the entirety of the pad 205 is contained between the labia of a wearer. FIG. 15 shows the manner in which the pad 205 is contained between the labia 18. The arrangement of this pad 205 is the same shape as the pad 20 of the first embodiment. The buckling strength of the peripheral parts of this pad 205 is no less than 0.1N and no more than 0.4N. Since the pad 205 thus moves in accompaniment with the movements of a wearer, the inner walls of the labia 18 will not become damaged even if the peripheral parts 265 contact the inner walls and the possibility of a wearer feeling a foreign-body sensation is also low.

[Buckling Strength]

Lastly, the buckling strength realized by the impact avoiding means (incisions in the case of the embodiments) shall now be described. The buckling strength is measured using an Instron Universal Material Testing Machine (made by Instron Japan Co., Ltd.).

<Measurement of the Buckling Strength of the Exposed Side Regions>

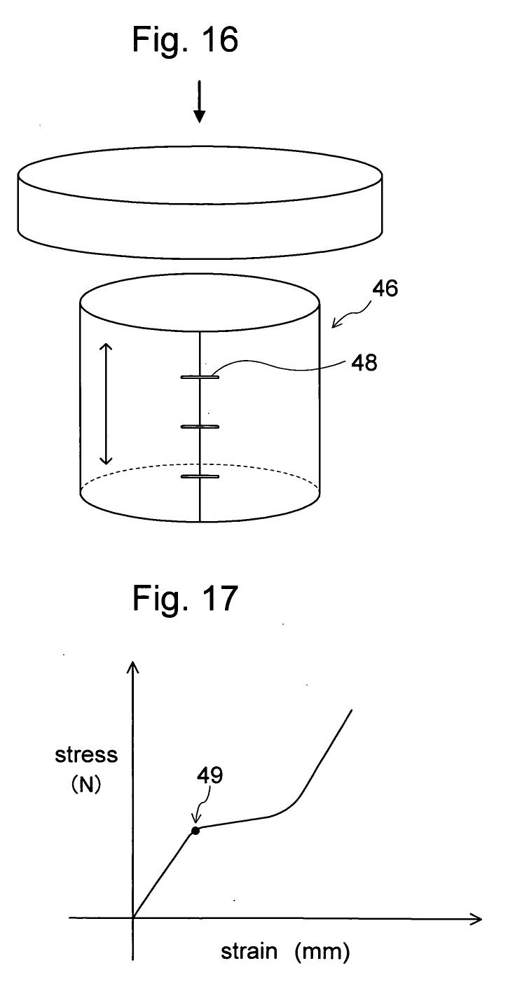

The absorbent body is used as the sample. As shown in FIG. 16, a sample 46 is formed to a cylindrical shape with a diameter of 45 mm and a height of 15 mm and is joined to itself using staples 48 at 5 mm intervals. The compressing speed of the tester is set to 30 mm/min and stress is applied from the top of the sample 46. The force that is applied in the longitudinal direction of the sample 46 is regarded to be the external force A and the external force B in FIG. 5. The relationship between the stress that is applied to the sample 46 and the strain is shown in FIG. 17. The stress at the first yield point 49 at the point at which the sample becomes compressed by 80% is determined as the buckling strength. This measurement is carried out five times and the average value thereof is determined as the buckling strength.

<Measurement of the Buckling Strength of the Peripheral Parts>

In regard to the sample, the surface side sheet and the back side sheet are used to form a cylindrical sample with a diameter of 45 mm and a height of 40 mm and measurements are made by the same procedures as those described above.

The buckling strength of the impact cushioning means comprises the buckling strength of the peripheral parts and the buckling strength of the exposed side regions. The peripheral parts may be formed by the surface side sheet and the back side sheet, by the surface side sheet, back side sheet, and the absorbent body sandwiched between the sheets, by only the surface side sheet, or by only the back side sheet. The exposed side regions are preferably arranged from the surface side sheet and the back side sheet. Since, among these, the member that influences the buckling strength the most is the absorbent body, the buckling strength of only the absorbent body is shown in Table 2. The measurement results are shown in Tables 1 and 2.

TABLE 1
Buckling strengths of external pressure cushioning means

| | | Sample 1 | Sample 2 | Sample 3 |
|---|---|---|---|---|
| Surface side | Raw material | 100% rayon | 100% rayon | 100% rayon |
| sheet | Basis weight | 35 | 35 | 35 |
| | (g/m²) | | | |
| | Manufacturing | Dry spun | Dry spun | Dry spun |
| | method | lacing | lacing | lacing |
| Back side | Raw material | 100% | 100% | 100% |
| sheet | | polyethylene | polyethylene | polyethylene |
| | Basis weight | 25 | 25 | 25 |
| | (g/m²) | | | |
| External | | Incisions + | Hot melt | Hot melt |
| pressure | | hot melt | | |
| cushioning | Basis weight | | 3.5 | 5.5 |
| means | (g/m²) | | | |
| | Pattern | Spray gun | Spray gun | Solid coating |
| | Pitch (mm) | 5 (length: | 2-5 | |
| | | 30 mm) | | |
| | Joined area | | 5 | 100 |
| | percentage (%) | | | |
| Buckling | | 0.19 | 0.3 | 0.44 |
| strength (N) | | | | |

Sample 1 in Table 1 has a hot melt applied by a spray gun and also has incisions formed therein, and the buckling strength of the impact cushioning means of the present embodiment is indicated thereby. Sample 2 has a hot melt applied by a spray gun and the buckling strength of the impact cushioning means of the second embodiment is indicated thereby. By Sample 3, the buckling strength of the peripheral parts of the prior-art interlabial pad described in Patent Document 1 is indicated. It can thus be understood that by using incisions as the impact cushioning means, external pressures can be cushioned efficiently.

TABLE 2
Buckling strengths of regions exposed from the labia and regions
contained between the labia (emboss area percentage: 1%)

| | Region exposed | | Region contained | |
|---|---|---|---|---|
| | from labia | | between labia | |
| | Sample | | | |
| | 1 | 2 | 1 | 2 |
| Raw material | 85% rayon + | 85% rayon + | 85% rayon + | 100% pulp |
| | 15% natural | 15% natural | 15% natural | |
| | cotton | cotton | cotton | |
| Basis weight | 180 | 360 | 180 | 120 |
| (g/m²) | | | | |
| Thickness (mm) | 3.5 | 5.5 | 4.5 | 4.5 |
| Density (g/cm³) | 0.05 | 0.07 | 0.07 | 0.07 |
| Buckling | 0.25 | 4.3 | 5.1 | 5.1 |
| strength (N) | | | | |

The buckling strengths of the samples in Table 2 are the buckling strength of the pad regions exposed from the labia and the pad regions contained in the labia of the respective embodiments. This Table shows that the buckling strength of the pad regions that are exposed from the labia (including the peripheral parts at which the impact cushioning means is provided) is preferably no less than 0.1N and no more than 5N. When the buckling strength is no more than 0.1N, the exposed pad regions become more readily compressed than necessary and body fluids that had been absorbed once will be readily discharged. On the other hand, if the buckling strength is greater than 5N, impacts from the groin parts 12 cannot be cushioned adequately. Also, the buckling strength of the part of the pad 20 that is contained between the labia is preferably no less than 2N and no more than 10N. When this buckling strength is less than 2N, the pad becomes readily twisted during fitting and gaps form between the pad and the inner labial walls. When the buckling strength is greater than 10N, there is a high possibility of making a wearer feel a foreign-body sensation.

1. An interlabial pad being applied by by inserting a part or whole thereof between labia,
the interlabial pad having, at least at peripheral parts that form the contour of the interlabial pad, a contact avoiding means avoiding contacts with a wearer's body that accompanies movements of the wearer in a fitted state; and/or an impact cushioning means cushioning the transmission of impacts resulting from contacts with the body.

2. The interlabial pad according to claim 1, wherein the interlabial pad has a substantially vertically long shape with a longitudinal direction and a lateral direction;
the contact avoiding means comprises waist parts formed by denting parts of both sides of the peripheral parts positioned at both sides of a longitudinal central axis extending along the longitudinal direction towards the longitudinal central axis; and,
in the fitted state, the waist parts are positioned at the narrowest position between legs at the groin part of the wearer.

3. The interlabial pad according to claim 1, wherein the impact cushioning means is arranged by a buckling strength of the peripheral parts being no less than 0.05N and no more than 0.4N.

4. The interlabial pad according to claim 1, wherein the impact cushioning means comprises a plurality of incisions that are provided in the interlabial pad.

5. The interlabial pad according to claim 4, wherein at least a part of the incisions are provided so as to be parallel to the lateral direction of the interlabial pad.

6. The interlabial pad according to claim 1, wherein the outer edges of the peripheral parts are wave-like in form.

7. The interlabial pad according to claim 1, wherein the interlabial pad is equipped with a cover member comprising a surface side sheet forming a surface side face that contacts the body in the filted state, and a back side sheet forming a back side face which is opposite to the surface side face; and
the impact cushioning means comprises discontinuously joined parts at which the surface side sheet and the back side sheet are joined discontinuously.

8. The interlabial pad according to claim 1, wherein the interlabial pad is equipped with a cover member comprising a surface side sheet forming a surface side face that contacts the body in the filted state, and a back side sheet forming a back side face at the side opposite the surface side face; and
the impact cushioning means includes microprotrusion regions formed of a set of microprotrusions disposed on the surface side sheet and/or the back side sheet.

9. The interlabial pad according to claim 8, wherein the mutual interval between the microprotrusions is no less than 0.3 mm and no more than 20 mm.

10. The interlabial pad according to claim 1, wherein the interlabial pad is equipped with a contained part which is held between the labia in the filted state, and exposed side regions which are exposed from at least one side of the labia; and
the contact avoiding means and/or the impact cushioning means are provided at the exposed side regions.

11. The interlabial pad according to claim 10, wherein the impact cushioning means is arranged by a buckling strength of the exposed side regions being no less than 0.1N and no more than 5.0N.

12. The interlabial pad according to claim 1, wherein the interlabial pad is used for absorbing vaginal discharge or for incontinence.
